(19) United States
(12) Patent Application Publication
(10) Pub. No.: US 2024/0268806 A1
(43) Pub. Date: Aug. 15, 2024

(54) Sealing Device for Closing a Large Bore Access Opening

(71) Applicant: Boston Scientific Scimed Inc., Maple Grove, MN (US)

(72) Inventors: Gary John Pederson, JR., Albertville, MN (US); Michael Nagel, Andover, MN (US); Andrew Roiko, Rogers, MN (US); Aaron Cooper Foss, Plymouth, MN (US)

(73) Assignee: Boston Scientific Scimed Inc., Maple Grove, MN (US)

(21) Appl. No.: 18/109,646

(22) Filed: Feb. 14, 2023

Related U.S. Application Data
(60) Provisional application No. 63/309,808, filed on Feb. 14, 2022.

(51) Int. Cl. A61B 17/00

(52) U.S. Cl. CPC A61B 17/0057; A61B 2017/00004; A61B 2017/00623; A61B 2017/00632; A61B 2017/00884

(57) ABSTRACT

Systems, devices, and methods for closing a large bore access site are disclosed. An example system may include a delivery catheter and a closure device releasably coupled to the delivery catheter. The closure device may include an expandable frame and a patch secured to the expandable frame. The closure device may be configured to shift between a delivery configuration and an expanded configuration. The closure device may be configured to be delivered by the delivery catheter through a blood vessel to a position adjacent to a large bore opening formed in the blood vessel while in the delivery configuration and then shifted to the expanded configuration within the blood vessel so that the patch engages and covers the large bore opening.

### CROSS-REFERENCE TO RELATED APPLICATIONS

This application claims the benefit of priority under 35 U.S.C. § 119 of U.S. Provisional Application No. 63/309,808, filed Feb. 14, 2022, the entire disclosure of which is hereby incorporated by reference.

### TECHNICAL FIELD

The present disclosure pertains to medical devices, and methods for manufacturing medical devices. More particularly, the present disclosure pertains to a device for closing a large bore access opening.

### BACKGROUND

A wide variety of intracorporeal medical devices have been developed for medical use, for example, intravascular use. Some of these devices include guidewires, catheters, closure devices, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A system for closing a large bore access site is disclosed. The system comprises: a delivery catheter; and a closure device releasably coupled to the delivery catheter, the closure device including an expandable frame and a patch secured to the expandable frame; wherein the closure device is configured to shift between a delivery configuration and an expanded configuration; wherein the closure device is configured to be delivered by the delivery catheter through a blood vessel to a position adjacent to a large bore opening formed in the blood vessel while in the delivery configuration and then shifted to the expanded configuration within the blood vessel so that the patch engages and covers the large bore opening.

Alternatively or additionally to any of the embodiments above, when the closure device is in the delivery configuration, the closure device has a delivery shape taking the form of a rolled cylinder with an axial overlapping region where opposing end regions of the closure device overlap.

Alternatively or additionally to any of the embodiments above, when the closure device is in the expanded configuration, the closure device has an expanded shape taking the form of a cylinder with an axial slot formed therein.

Alternatively or additionally to any of the embodiments above, the expandable frame comprises a stent.

Alternatively or additionally to any of the embodiments above, the expandable frame comprises a nickel-titanium alloy.

Alternatively or additionally to any of the embodiments above, the expandable frame comprises a bioabsorbable material.

Alternatively or additionally to any of the embodiments above, the patch comprises a biodegradable material.

Alternatively or additionally to any of the embodiments above, the patch comprises one or more substances that promote tissue-healing.

Alternatively or additionally to any of the embodiments above, the expandable frame comprises a spine region having a retrieval member coupled thereto.

Alternatively or additionally to any of the embodiments above, the expandable frame has a plurality of angled ribs extending from the spine region.

A method for closing a large bore access site is disclosed. The method comprises: advancing a closure device through a blood vessel using a delivery device from a first location spaced apart from a large bore opening formed in the blood vessel to a second location adjacent to the large bore opening, the closure device including an expandable framework and a patch coupled to the expandable framework; deploying the closure device; and wherein deploying the closure device includes expanding the closure device against an inner wall surface of the blood vessel and into contact with the large bore opening.

Alternatively or additionally to any of the embodiments above, further comprising aligning the closure device with the large bore opening.

Alternatively or additionally to any of the embodiments above, wherein aligning the closure device with the large bore opening includes rotating the delivery device.

Alternatively or additionally to any of the embodiments above, further comprising forming a small bore opening in the blood vessel adjacent to the first location and advancing the delivery device through the small bore opening.

Alternatively or additionally to any of the embodiments above, wherein advancing a closure device through a blood vessel includes advancing the closure device in a delivery configuration through the blood vessel; and wherein when the closure device is in the delivery configuration, the closure device has a delivery shape taking the form of a rolled cylinder with an axial overlapping region where opposing end regions of the closure device overlap.

Alternatively or additionally to any of the embodiments above, wherein deploying the closure device includes shifting the closure device to an expanded configuration; wherein when the closure device is in the expanded configuration, the closure device has an expanded shape taking the form of a cylinder with an axial slot formed therein.

Alternatively or additionally to any of the embodiments above, further comprising retrieving the closure device from the blood vessel.

A device for closing a large bore access site is disclosed. The device comprises: an expandable frame; and a patch secured to the expandable frame; wherein the closure device is configured to shift between a delivery configuration and an expanded configuration; wherein the closure device is configured to be delivered by a delivery catheter through a blood vessel from a remote location to a position adjacent to a large bore opening formed in the blood vessel while in the delivery configuration and then shifted to the expanded configuration within the blood vessel so that the patch engages and covers the large bore opening.

Alternatively or additionally to any of the embodiments above, the expandable frame comprises a spine region having a retrieval member coupled thereto.

Alternatively or additionally to any of the embodiments above, the expandable frame has a plurality of angled ribs extending from the spine region.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of an example closure device.

FIG. 2 is an end view of a perspective view of an example closure device in a generally flat configuration.

FIG. 3 is an end view of a perspective view of an example closure device in a rolled configuration.

FIG. 4 is a perspective view of an example closure device.

FIG. 5 schematically depicts a large bore opening.

FIG. 6 illustrates an example delivery device/catheter.

FIGS. 7-9 illustrate an example method for delivering an example closure device.

FIG. 10 is a perspective view of an example closure device.

FIG. 10A is a perspective view of an example closure device.

FIG. 11 is a perspective view of an example closure device.

FIG. 12 depicts an example closure device in a generally flat configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

A number of intravascular interventions include the formation of a puncture into a blood vessel to provide an access opening for a catheter or the like to enter the vasculature. The size of the opening is dictated by the size of the catheter being used for the intervention. For example, an intervention that utilizes a relatively small catheter for delivering a coronary stent may only need a relatively small access opening whereas an intervention that utilizes a larger catheter for delivering a larger device (e.g., such as a replacement heart valve) may need a larger opening.

A number of practices are utilized to close the access opening after the intervention. For example, in some instances manual compression may be used to close the opening. In other instances, an access closure device may be used to close the access opening. There is an ongoing need for new and different closure devices, particularly for larger access openings such as those made during an intervention that utilizes a larger device.

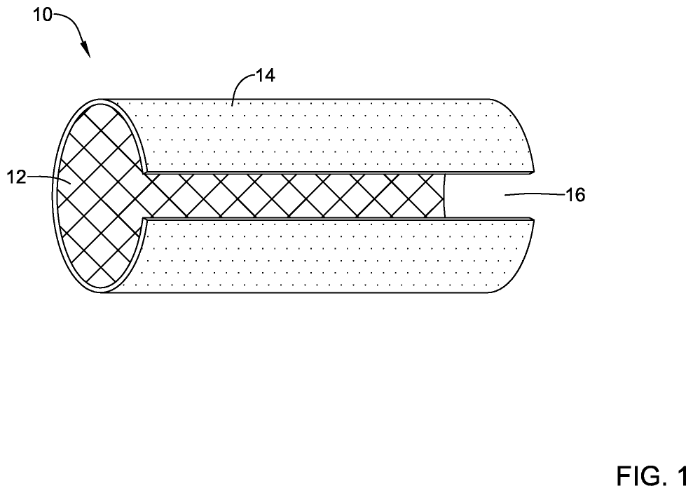

FIG. 1 is a perspective view of an example closure device 10. The closure device 10 may include an expandable frame 12 and a patch or fabric 14 coupled to the expandable frame 12. The expandable frame 12 may have any one of a variety of forms/configurations. For examples, in some instances, the expandable frame 12 may have geometric shape (e.g., such as a square or rectangle when the expandable frame 12 is seen in a flattened or planar configuration) that extends around the periphery of the closure device 10. It can be appreciated that a variety of geometric shapes may be utilized (e.g., polygonal, regular polygonal, irregular polygonal, regular shapes, irregular shapes, etc.). In other examples, the expandable frame 12 may have a mesh or stent-like configuration. This may include one or more filaments that cross one another (e.g., similar to a braid) or a framework with one or more cutouts or openings formed therein. In some instances, the expandable frame 12 may take the form of or resemble a stent.

The patch or fabric 14 may take the form of a tape or membrane that is secured to the expandable frame 12. In general, the patch or fabric 14 is designed to be pressed up against or into contact with a large bore access opening. In some instances, the patch or fabric 14 may include one or more substances that promote tissue healing or that otherwise help to expedite the healing of the large bore access opening when brought adjacent to or into contact with the opening. In at least some instances, the patch or fabric 14 may include a bioabsorbable or bioerodible material. In such instances, when the closure device 10 is delivered/deployed, the patch or fabric 14 would bioabsorb or bioerode over time, leaving behind the expandable frame 12 (e.g., which may be capable of being visualized using fluoroscopy in subsequent interventions). In at least some instances, the expandable frame 12 may include a bioabsorbable or bioerodible material. In still other instances, both the expandable frame 12 and the patch or fabric 14 may be formed from a bioabsorbable or bioerodible material so that both would bioabsorb or bioerode over time. Some examples of bioabsorbable and/or bioerodible materials may include poly(lactide-co-glycolide) (PLGA), poly(D,L-lactic acid) (PDLA), poly(L-lactic acid) (PLLA), poly(caprolactone) (PCL), polyhydroxy-butyrate/-valerate copolymer (PHBV), polyorthoester (POE), polyethyleneoxide/polybutylene terephthalate copolymer (PEO/PBTP), one or more polyamides (such as Nylon 66 and polycaprolactam), one or more polyanhidride, magnesium (and/or other bioabsorbable/bioerodible metals), combinations thereof, and/or the like.

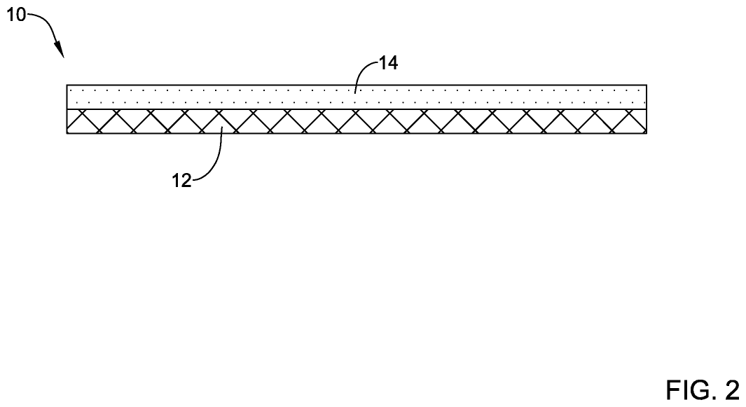

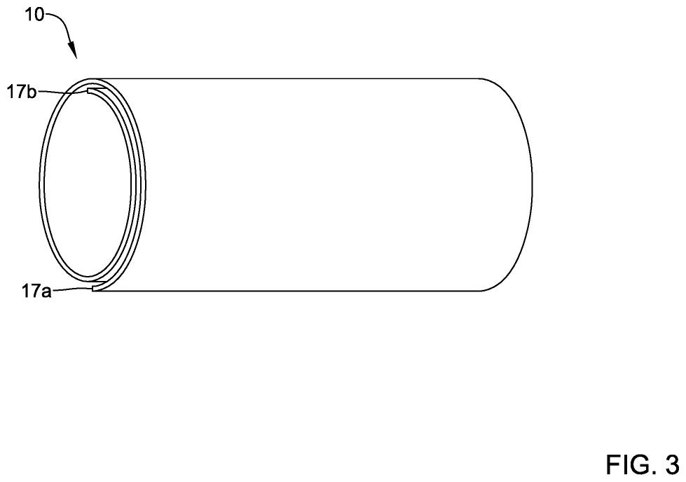

The closure device 10 may be designed and/or configured to take a variety of shapes and/or configurations. For example, as depicted in FIG. 1, the closure device 10 may be configured to shift to or otherwise take a cylindrical form or shape. When doing so, an axial slot 16 may be defined along a side of the closure device 10. Such a shape may be understood to be a deployed or delivered shape. In some instances, the expandable frame 12 may be formed from a material that has a shape memory and/or super-elasticity (e.g., a nickel-titanium alloy such as NITINOL) that allows the closure device 10 to take the deployed shape when delivered within a body lumen. Other shapes/configurations are contemplated. For example, as depicted in FIG. 2, the closure device 10 may also be designed and/or configured to take a flat or generally planar form or shape. In some instances, the planar shape may be utilized or seen during manufacturing of the closure device 10 or seen when then expandable frame 12 is at a temperature below its pre-set shape memory temperature (e.g., when the expandable frame 12 is formed from a shape memory material).

While in some instances the expandable frame 12 may be formed or otherwise configured to be self-expanding, in other instances the expandable frame 12 may be formed from or otherwise be configured to be expanded using an expandable member or balloon. In such instances, the expandable frame 12 may include balloon-expandable materials (e.g., metals, metal alloys, and/or the like) and/or otherwise resemble a balloon-expandable stent.

As depicted in FIG. 3, the closure device 10 may also be designed and/or configured to take a rolled or compressed cylindrical form or shape. In this example, the opposing end regions 17a, 17b of the closure device 10 overlap with one another, essentially closing or filling the slot 16 (e.g., which is not labeled in FIG. 3). This may also be described as a rolled cylinder with an axial overlapping region where opposing end regions 17a, 17b of the closure device 10 overlap. The rolled or compressed form may be understood to be a delivery configuration suitable for delivering the closure device 10 a large bore access opening.

In general, the closure device 10 is configured to engage the blood vessel at or adjacent to a large bore access opening. This may include delivering the closure device 10 through the blood vessel while in a first or delivery configuration (e.g., as shown in FIG. 3) via a delivery catheter (e.g., as discussed in herein). While being delivered, the closure device 10 may be held or constrained in the delivery configuration. Upon reaching the target site at or adjacent to the large bore access opening, the delivery catheter can be actuated to allow the closure device 10 to shift from the delivery configuration to a second or expanded configuration (e.g., as shown in FIG. 1). This may include the expandable frame 12 expanding or self-expanding so as to shift the closure device 10 to the expanded configuration. When doing so, the expandable frame 12 may help to press the closure device 10 against the large bore access opening (e.g., which may also be described as pressing the patch or fabric 14 against the large bore access opening) so that the large bore access opening can be closed.

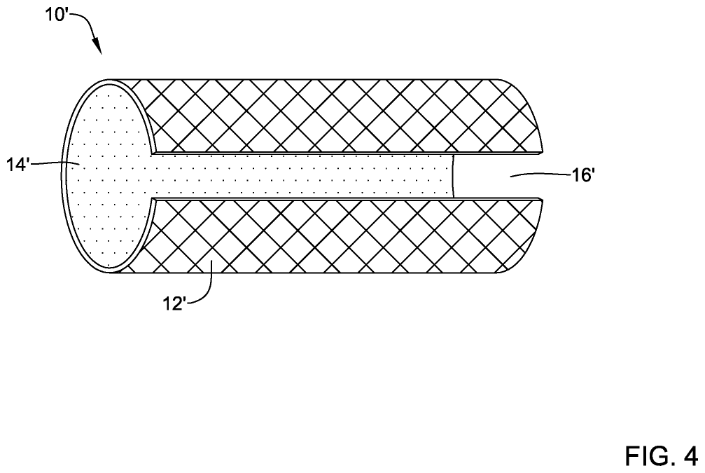

In some instances, the expandable frame 12 may be understood to be disposed along an interior or inner surface of the closure device 10 and the patch or fabric 14 may be disposed along an exterior or outer surface of the closure device 10. This is not intended to be limiting. For example, FIG. 4 illustrates another example closure device 10′, similar in form and function to the closure device 10, where the expandable frame 12′ may be disposed along an exterior or outer surface of the closure device 10′ and the patch or fabric 14′ may be disposed along an interior or inner surface of the closure device 10′.

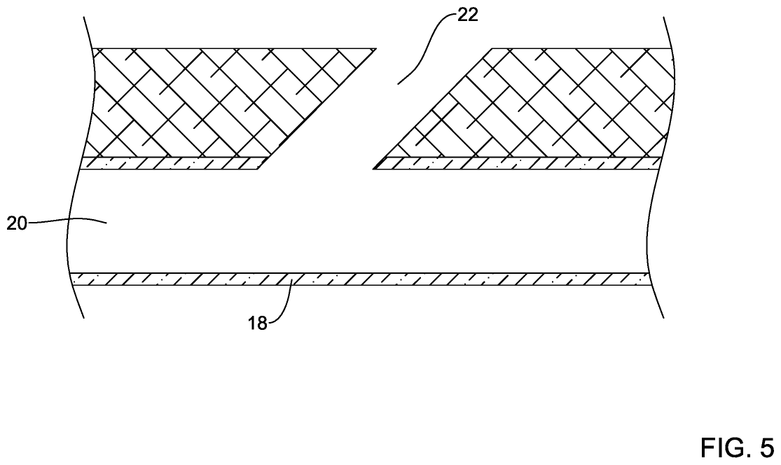

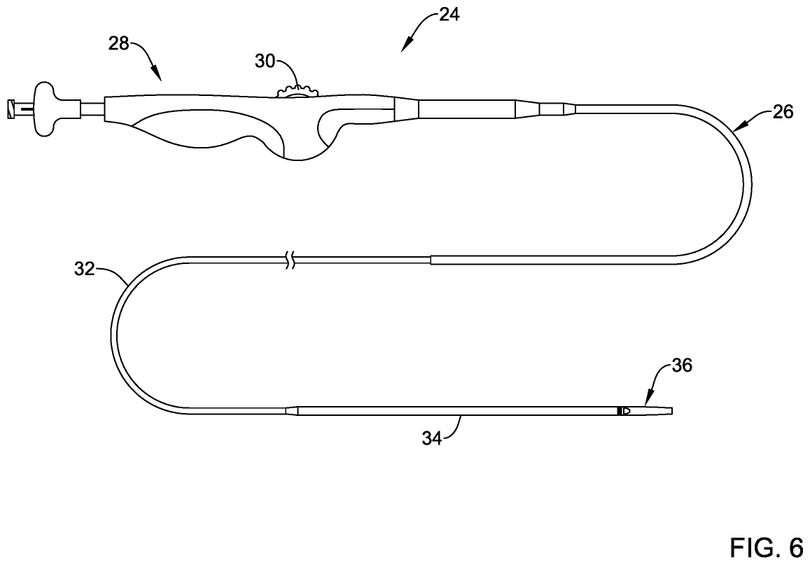

FIG. 5 schematically illustrates an example blood vessel 18 having a lumen 20 formed therein and a large bore access opening 22. As indicated above, the closure device 10 may be used to close the large bore access opening 22. In order to close the large bore access opening 22, the closure device 10 may be delivered to the large bore access opening 22 from another location within the blood vessel 18. This may include forming another, smaller opening into the blood vessel at a location positioned apart from the large bore access opening 22 and delivering the closure device 10 to the large bore access opening 22 using a suitable delivery catheter such as the delivery device/catheter 24 depicted in FIG. 6. Because only a smaller opening is needed for the delivery catheter 24 to access the blood vessel 18 (e.g., in order to close the large bore access opening 22 from within the blood vessel 18), it may be possible for a clinician to later seal the small opening using traditional methods such as manual compression.

The delivery catheter 24, in general, may include a shaft or shaft assembly 26 having a handle 28 coupled thereto. An actuator 30 may be coupled to the handle 28. The shaft or shaft assembly 26 may include a distal tip region 36. In at least some instances, the actuator 30 may be designed to retract a deployment sheath 32 so that a device covering region 34 of the deployment sheath 32 uncovers the closure device 10. The delivery catheter 24 shown is meant to be an example. In some instances, the delivery catheter 24 may include features of the catheters disclosed in U.S. Pat. Nos. 8,784,468, 9,084,692, and 9,220,619, the entire discloses of which are herein incorporated by reference.

The delivery catheter 24 depicted in FIG. 6 illustrates an example device that may be used to deliver the closure device 10 when, for example, the expandable frame 12 includes a shape memory and/or super elastic material. In addition, it can be appreciated that in some instances, the expandable frame 12 may include balloon-expandable materials (e.g., metals, metal alloys, and/or the like) and/or otherwise resemble a balloon-expandable stent. In these instances, the delivery catheter 24 may take the form of a catheter with an expandable member or balloon that is capable of delivering and expanding the closure device 10 at a suitable target location. Accordingly, the closure device 10 may be loaded upon a balloon catheter (e.g., onto a folded balloon of the balloon catheter) and delivered to the large bore access opening 22 and deployed using the balloon catheter delivery device.

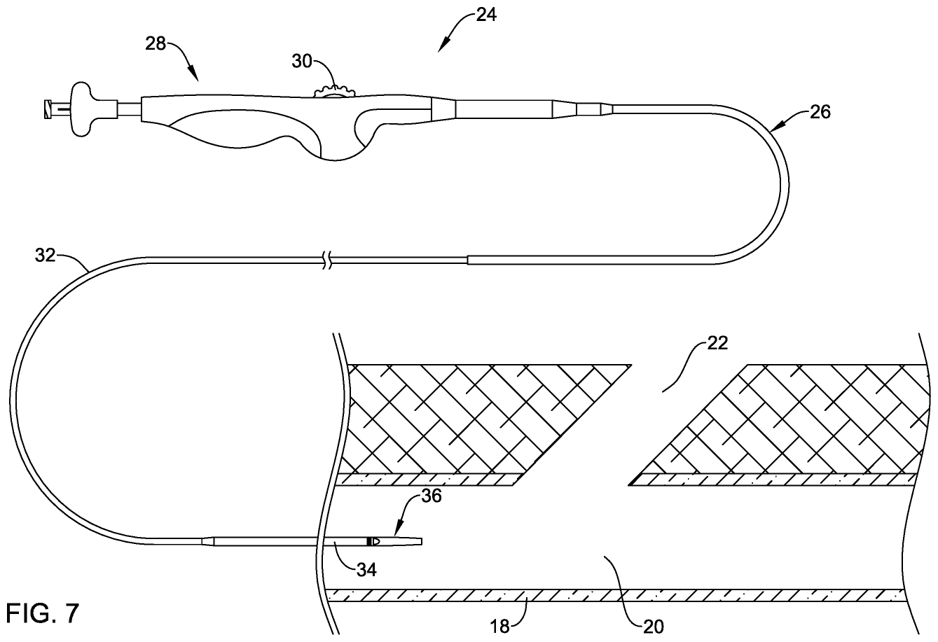

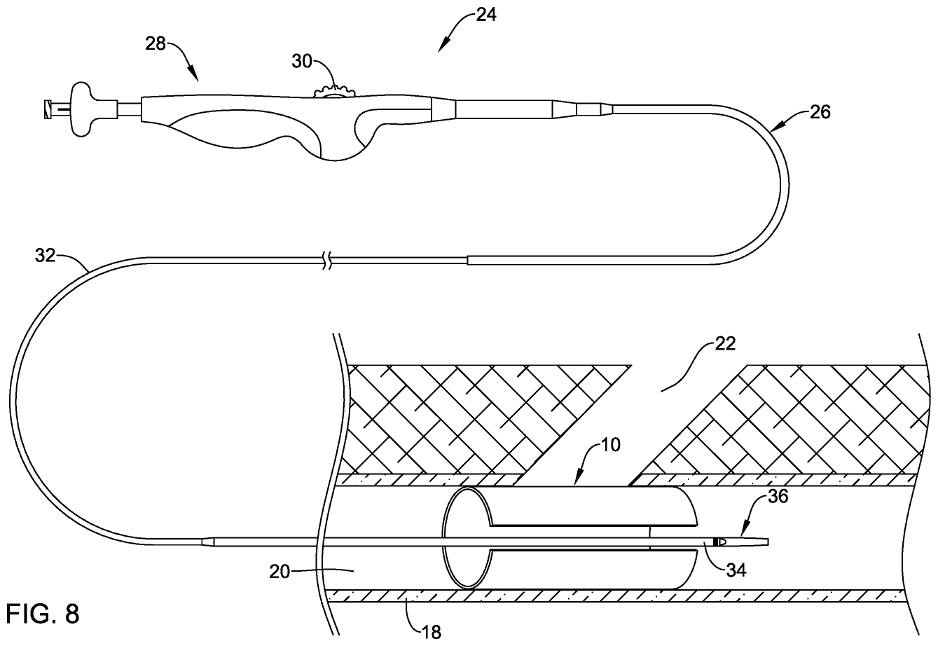

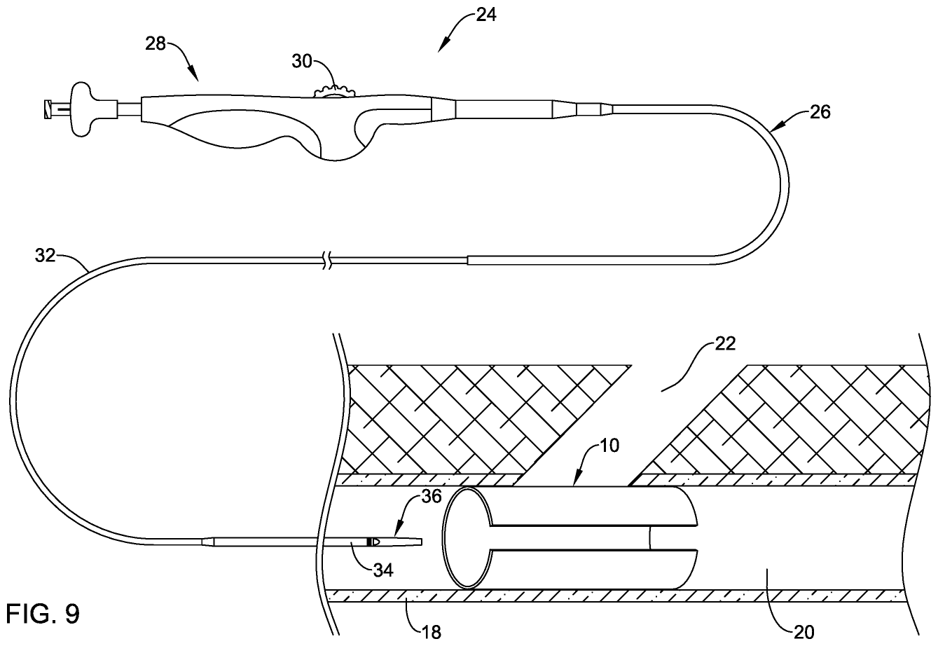

FIGS. 7-9 depict an example method for delivering/deploying the closure device 10. For example, the delivery catheter 24 may be navigated through the blood vessel 18 from a remote location to a position adjacent to the large bore access opening 22 as shown in FIG. 7. The distal tip region 36 may be aligned with or otherwise navigated just past the large bore access opening 22 and the deployment sheath 32 may be retracted to deploy the closure device 10 as shown in FIG. 8. In some instances, the delivery catheter 24 can be moved, translated, rotated, and/or other aligned with the opening 22 to best place the closure device 10 at the desired target location. When the closure device 10 is suitably deployed, delivery catheter 24 can be removed from the blood vessel 18 as shown in FIG. 9.

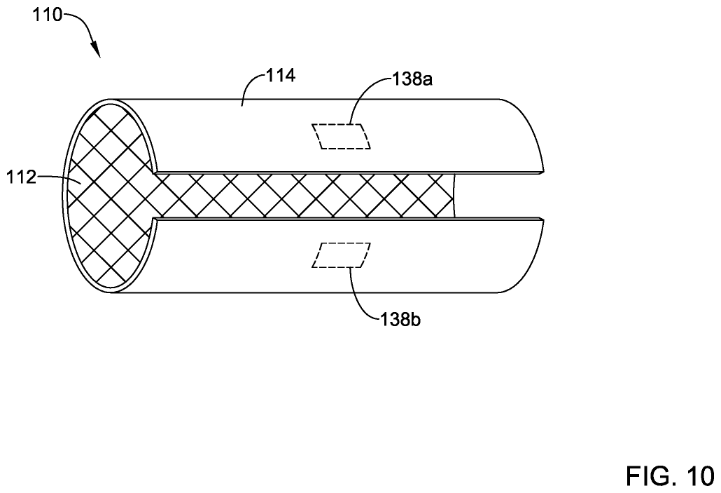

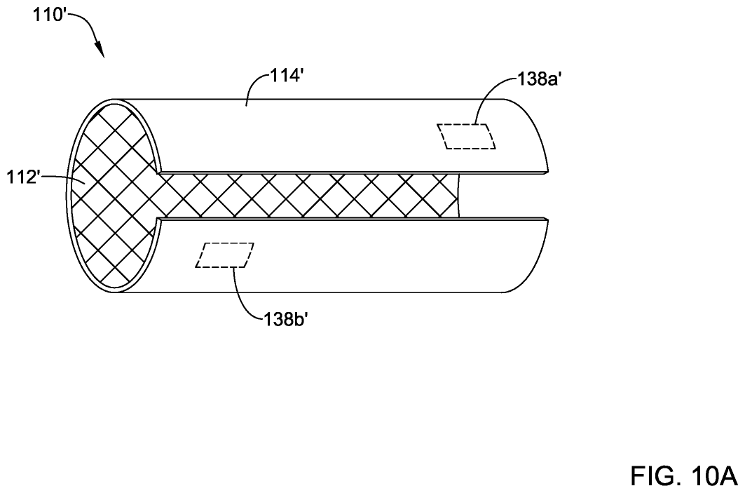

FIG. 10 illustrates another example closure device 110 that may be similar in form and function to other closure devices disclosed herein. The closure device 110 may include an expandable frame 112 and a patch or fabric 114 coupled to the expandable frame 112. In this example, the closure device 110 may include one or more alignment members 138a, 138b. In general, the alignment members 138a, 138b may be utilized to help aid a clinician in deploying the closure device 110 in a desired manner. For example, the alignment members 138a, 138b may take the form of radiopaque markers that allow a clinician to visualize the closure device 110 via fluoroscopy during a closure procedure. The known position(s) of the alignment members 138a, 138b allow for the clinician to know whether the closure device 110 is rotationally aligned with the large bore access opening. For example, if the alignment members 138a, 138b are disposed on opposite sides of the closure device 110, a clinician may see either two distinct marks corresponding to the alignment members 138a, 138b or a singular mark (e.g., when the alignment members 138a, 138b are aligned with the line of sight). Other configurations and arrangements of the alignment members 138a, 138b are contemplated. For example, FIG. 10 depicts the alignment members 138a, 138b generally positioned adjacent to the middle of the closure device 110. FIG. 10A depicts a closure device 110′ (e.g., including an expandable frame 112′ and a patch or fabric 114′ coupled to the expandable frame 112′) where the alignment members 138a′, 138b′ are disposed closer to the end regions of the closure device 110′. It can be appreciated that a number of arrangements are contemplated for the location of various alignment members (e.g., adjacent to the middle, adjacent to one or more of the ends, combinations thereof, etc.).

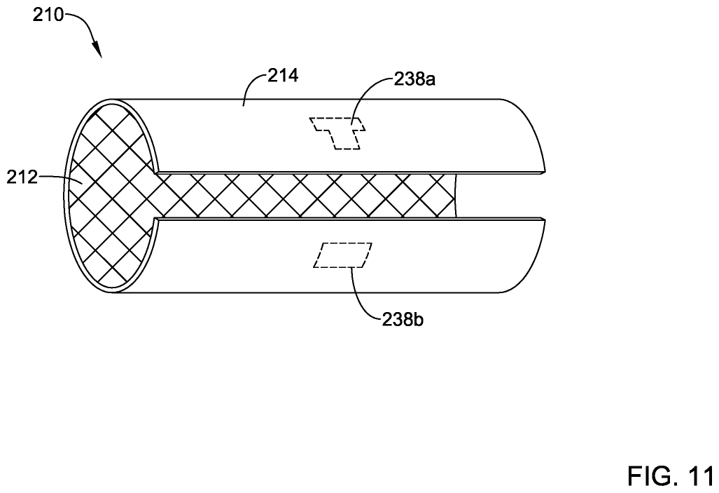

FIG. 11 illustrates another example closure device 210 that may be similar in form and function to other closure devices disclosed herein. The closure device 210 may include an expandable frame 212 and a patch or fabric 214 coupled to the expandable frame 212. One or more alignment members 238a, 238b may be disposed along the closure device 110. In general, the alignment members 238a, 238b may be utilized to help aid a clinician in deploying the closure device 210 in a desired manner. In this example, the alignment members 238a, 238b may have dissimilar shapes. The distinct/different shapes, along with the known position(s) of the alignment members 238a, 238b allow for the clinician to know whether the closure device 210 is rotationally aligned with the large bore access opening.

In some instances, the delivery catheter 24 may also include one or more alignment members. Such alignment members may be aligned with the alignment members of an example closure device (e.g., the closure device 110, 110′, 210). Alternatively, the alignment members on the delivery catheter 24 may be offset from the alignment members of an example closure device (e.g., the closure device 110, 110′, 210). Alignment members on the delivery catheter 24 may aid a clinician in visualizing the orientation of the delivery catheter 24 and/or the closure device during an intervention.

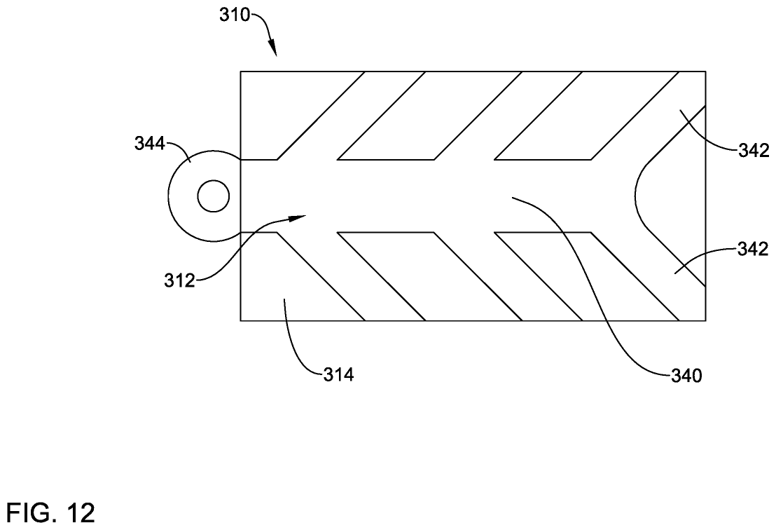

FIG. 12 illustrates another example closure device 310 that may be similar in form and function to other closure devices disclosed herein. The closure device 310 may include an expandable frame 312 and a patch or fabric 314 coupled to the expandable frame 312. In this example, the expandable frame 312 may include a spine or spine region 340 and a plurality of ribs or spokes 342, which may be angled, extending from the spine region 340.

The closure device 310 may also include a retrieval member 344. The retrieval member 344 may take the form of a graspable projection extending from the expandable frame 312 (and/or extending from the closure device 310, in general) that can be accessed by a separate retrieval device. Such a retrieval device (not shown) may be capable of engaging the retrieval member 344 so that the expandable frame 312 can be retrieved/removed from the blood vessel. In some instances, the retrieval member 344 may include an opening or eyelet, which may help a retrieval device to securely engage the retrieval member 344.

The materials that can be used for the various components of the closure device 10 (and/or other closure devices disclosed herein) may include those commonly associated with medical devices. For example, the closure device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the closure device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the closure device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the closure device 10 to achieve the same result.

In some embodiments, a degree of Magnetic Resonance Imaging (MRI) compatibility is imparted into the closure device 10. For example, the closure device 10, or portions thereof, may be made of a material that does not substantially distort the image and create substantial artifacts (e.g., gaps in the image). Certain ferromagnetic materials, for example, may not be suitable because they may create artifacts in an MRI image. The closure device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for closing a large bore access site, the system comprising:
a delivery catheter; and
a closure device releasably coupled to the delivery catheter, the closure device including an expandable frame and a patch secured to the expandable frame;
wherein the closure device is configured to shift between a delivery configuration and an expanded configuration;
wherein the closure device is configured to be delivered by the delivery catheter through a blood vessel to a position adjacent to a large bore opening formed in the blood vessel while in the delivery configuration and then shifted to the expanded configuration within the blood vessel so that the patch engages and covers the large bore opening.

2. The system of claim 1, wherein when the closure device is in the delivery configuration, the closure device has a delivery shape taking the form of a rolled cylinder with an axial overlapping region where opposing end regions of the closure device overlap.

3. The system of claim 1, wherein when the closure device is in the expanded configuration, the closure device has an expanded shape taking the form of a cylinder with an axial slot formed therein.

4. The system of claim 1, wherein the expandable frame comprises a stent.

5. The system of claim 1, wherein the expandable frame comprises a nickel-titanium alloy.

6. The system of claim 1, wherein the expandable frame comprises a bioabsorbable material.

7. The system of claim 1, wherein the patch comprises a biodegradable material.

8. The system of claim 1, wherein the patch comprises one or more substances that promote tissue-healing.

9. The system of claim 1, wherein the expandable frame comprises a spine region having a retrieval member coupled thereto.

10. The system of claim 9, wherein the expandable frame has a plurality of angled ribs extending from the spine region.

11. A method for closing a large bore access site, the method comprising:
advancing a closure device through a blood vessel using a delivery device from a first location spaced apart from a large bore opening formed in the blood vessel to a second location adjacent to the large bore opening, the closure device including an expandable framework and a patch coupled to the expandable framework;
deploying the closure device; and
wherein deploying the closure device includes expanding the closure device against an inner wall surface of the blood vessel and into contact with the large bore opening.

12. The method of claim 11, further comprising aligning the closure device with the large bore opening.

13. The method of claim 12, wherein aligning the closure device with the large bore opening includes rotating the delivery device.

14. The method of claim 11, further comprising forming a small bore opening in the blood vessel adjacent to the first location and advancing the delivery device through the small bore opening.

15. The method of claim 11, wherein advancing a closure device through a blood vessel includes advancing the closure device in a delivery configuration through the blood vessel; and
wherein when the closure device is in the delivery configuration, the closure device has a delivery shape taking the form of a rolled cylinder with an axial overlapping region where opposing end regions of the closure device overlap.

16. The method of claim 15, wherein deploying the closure device includes shifting the closure device to an expanded configuration;
wherein when the closure device is in the expanded configuration, the closure device has an expanded shape taking the form of a cylinder with an axial slot formed therein.

17. The method of claim 11, further comprising retrieving the closure device from the blood vessel.

18. A device for closing a large bore access site, the device comprising:
an expandable frame; and
a patch secured to the expandable frame;
wherein the closure device is configured to shift between a delivery configuration and an expanded configuration;
wherein the closure device is configured to be delivered by a delivery catheter through a blood vessel from a remote location to a position adjacent to a large bore opening formed in the blood vessel while in the delivery configuration and then shifted to the expanded configuration within the blood vessel so that the patch engages and covers the large bore opening.

19. The device of claim 18, wherein the expandable frame comprises a spine region having a retrieval member coupled thereto.

20. The device of claim 19, wherein the expandable frame has a plurality of angled ribs extending from the spine region.
